(19) United States
(12) Patent Application Publication
(10) Pub. No.: US 2019/0006056 A1
(43) Pub. Date: Jan. 3, 2019

(54) SENSING MATERIAL AND UNIT FOR SENSING PHYSIOLOGICAL PARAMETER AND METHOD FOR PRODUCING SENSING MATERIAL UNIT

(71) Applicant: MAKALOT INDUSTRIAL CO., LTD., Taipei (TW)

(72) Inventors: Chih-Wei Chiu, Taipei (TW); Chen-Yang Huang, Taipei (TW); Wei-Yi Tsai, Taipei (TW)

(73) Assignee: Makalot Industrial Co., LTD., Taipei (TW)

(21) Appl. No.: 15/637,083

(22) Filed: Jun. 29, 2017

(51) Int. Cl. H01B 1/04; A61B 5/00

(52) U.S. Cl. CPC H01B 1/04; A61B 5/02141; A61B 5/0006

(57) ABSTRACT

A sensing material for sensing a physiological parameter includes a carbon black material, a graphene material and a glue material, wherein the carbon black material, the graphene material and the glue material are mixed together based on a specific weight ratio.

### CROSS REFERENCE TO RELATED APPLICATION

This application claims the benefit of the Taiwan Patent Application No.105135790, filed on Nov. 3, 2016, at Taiwan Intellectual Property Office, the disclosures of which are incorporated herein in their entirety by reference.

### TECHNICAL FIELD

Embodiments in the present disclosure are related to a sensing material for sensing a physiological parameter, a unit for sensing a physiological parameter and a method for producing a sensing material unit, especially related to a non-metal conductive material, which can be applied to the sensing material for sensing a physiological parameter with an organism.

### BACKGROUND

In recent years, wearable devices have become one of the most popular products in the science and technology field, and the technology applied to smart wear products in 2016 received a lot of attention in the media. In addition to updates on familiar products, such as an Apple® smart watch, a Google® smart glass, a LG® smart hand ring and a Samsung® smart hand ring, more diverse types of wearable devices have sprung up and been developed, such as a necklace, an ear ring, jewelry, clothes, etc. The purse pose and fields to which the most recent developments have been applied is also becoming more diverse, such as the information entertainment, exercising, fitness, medical care, and so on. Such products are being further combined with application programs, and they not only to provide users with more functions and services, but also create a new business opportunities for the wearable device manufactures. However, all the currently available wearable devices have a common problem, that is, the sensors have limited functionality, resulting in only acquiring a limited range of human body messages, thus causing unstable signals because they are not closely touching to the skin.

The prototype of the smart wearable cloth was invented by Professor Sundaresan Javaraman and Dr. Sung-Mee Park in George Institute of Technology sponsored by the US army, and was developed as “Georgia Tech Wearable Motherboard”, which can detect the heath condition of soldiers in the battle field. The smart wearable cloth receives and transmits the vital signs by using a metal fabric material, and has taken the lead to be a patented product.

In the development of the smart wearable clothing field, Adidas, has brought out a product called “Men's Training Shirt” which can detect the vital signs, such as the heart beat, the training intensity and the calorie consumption. Another product called “Speed cell motion detection” attached to sports shoes can record speed and distance data which can be uploaded to an application program through a Bluetooth® communication for monitoring.

X Cell's motion detection and vital signs detection can track the vertical height, the agility and the heart rate index during exercising and training, and upload these data to the application program by a wireless communication for monitoring purposes. Athos®, a company, has brought out smart clothing, which has such functions as an electro-myographic recording (EMG), and recordings of target heart rhythm, average rhythm, breathing, calorie consumption, etc. There are twenty six sensors on the shirt and pants that can monitor all the different data.

OMSignal, a Canadian company, employs specialized staffs including a neuroscientist, a sports medical expert, an engineer, etc, and has developed a T-shirt sensor that can count user's walking steps, breath, heart beats, and can send an alarm signal to the user when an abnormal value is detected. Its product is called “SMART BOX”, and it can last 30 hours from one full charge, and it sweat and rain resistant. However, it is not fully waterproof.

In addition, there are research centers for smart clothing in Taiwan, such as the Institute of Textile Industry, Fong-Jia university and Nan-Wei Industrial Co., Ltd. Textile Research Institute in Taiwan and Wan-Jiou technology company are also cooperating to develop material for smart clothing. The Wan-Jiou technology company is a company that is developing and manufacturing a wireless device product with a heart beat sensor and sports monitor product for sports; and its parent company is a medical equipment company in England. Wan-Jiou is investing fifty million NT dollars to obtain the relevant license and technology transfer from the Textile Research Institute, and is currently developing professional manufacturing equipment. The main technology that the Textile Research Institute manufactures is “fabric electros” which was silver fibers characterized as breathable, hygroscopic, soft, resistant to twisting, and which can also pass the AATCC135 100 times washable test. The fabric electrodes, conductive materials, connectors and other components are integrated in a conductive ribbon: as long as the conductive ribbon is purchased, the conductive ribbon is attached to the sports apparel by means of sewing, and then the heart rate detection can be performed.

In order to meet the demand of the practical application, more research on the intelligent clothing will improve the present drawbacks of such wearable devices. The sports brand “Goldwain” was responsible for the design and production in 2014, and then later three companies including Japanese telecommunications giant, NTT and DOCOMO cooperated to develop the functional material “Hitoe” sports T-shirt, combined with sensor chips and fabric cloth. The “Hitoe” sports T-shirt can be used to record a state of a body by using the display of the mobile phone, and is a smart sports material that can measure a heartbeat and a pulse, and show an electrocardiogram (ECG). Among these smart apparel companies, Nan-Wei Industrial co., Ltd. won the German IF product design award in 2014. The wearable human health devices for sale are for the most part focused on sports and heart rate detection of medical patients. AiQ Smart Clothing Technology Company has broken away from the traditional concept of wearing a strap and chest belt type heath monitors, so that consumers can really wear the health device as an item of clothing. Smart clothing technology covers the textile, materials, electronic and medical fields, and the metal fiber technology therein is the company's core technology, as shown in FIG. 1.

Please refer to FIG. 1, which shows a conductive material of an inner layer of a smart cloth in the prior art. In Taiwan's Patent No. 1336738 reference, the fabric 10 includes a fabric structure 12 including a first fabric layer 102, a second fabric layer 104, an insulating layer 108 and conductive yarn 120 with which the first to the second fabric layer are interwoven. The conductive yarn 120 includes a detection region 120a and a conductive region 120b. The crest 120c of the detection region 120a protrudes from one side of the first fabric layer 102, thereby contacting the skin to sense a physiological parameter, and the trough 120d of the detection region 120a is interwoven in the first fabric layer 102. The conductive yarn 120 consists of a plurality of conductive fibers 123, and each conductive fiber 123 includes a metal plating layer 123a and a fiber layer 123b. The conductive yarn 120 is made to have a bent shape or a corrugated shape in order to increase the elasticity of the metallic material and to prevent wearers feeling wearing uncomfortable due to the poor elasticity and stretch ability of the conductive yarn 120 when the conductive fiber 123 is plated with the metal. In addition, the conductive yarn 120 must also be intertwined with the the fabric layers 102, 104 to increase their elasticity, which results in a complex process that causes the yield to decrease. For example, by first spinning the conductive yarns 120 having the metal material, the conductive yarns 120 are carefully twisted into the desired convex shape, and then intertwined with the resilient insulating yarn 122 and 124, which is time consuming and cumbersome.

At present, well-known domestic and foreign sport apparel companies continue to invest in the sale of smart clothing products, but material used for signal reception and transmission is mainly metal conductive yarn, and metal materials are not flexible. In order to increase the flexibility, the production process will be complex. It is desirable to have a wearable sensor material that can be both flexible and have a good sensing effect.

### SUMMARY OF EXEMPLARY EMBODIMENTS

This technology is mainly to produce low-cost, easily processed, washable conductive slurry with high conductivity, that can further be made into a thin film material, and then the film material is applied to the smart clothing. This technology solves problems such as smart clothing made by metal fiber is non-washable and expensive in the general market. Smart clothing can be combined with an ECG detecting device to produce a stable signal to sense consumer's heartbeat, heart rhythm and other physical information, which can be applied to myriad uses in the sport and medical field. The carbon material used in the present invention is mainly to replace metal material and so be harmless to the human body, and to serve as a material for the sensing unit while increasing flexibility and comfort during wearing.

The polymers are covalently bonded to each other to form molecule sub-segments, but the electrons of the chemical bonds formed by them can not move, and the molecules are inactive, so the polymers are usually electrically neutral. In general, the polymer itself is an insulator, but can be changed in the molecular structure or doped with conductive particles to make it possible to have a conductive effect, known as a conductive polymer. The conductive polymer can be classified as an intrinsic conductive polymer or a composite conductive polymer according to its different conductive mechanism.

The intrinsic conductive polymer (ICP) is formed by an alternating single and double conjugate bond, and by using the adjacent unpaired electron cloud, can cross over the energy gap to achieve the purpose of conduction. However, intrinsic conductive polymers have positive electricity and negative electricity to attract so as to be condensed into a larger particle, which results in extreme difficulty in manufacturing applications.

The composite conductive polymer (ECP) is based on a polymer substrate, which is doped with conductive particles to achieve the purpose of conduction by means of the physical composite method. In order to improve the conductivity, the conductive particles must have good conductivity, must not to drift, and have no effect to the environment. The conductive particles often used are listed in the following categories: (1) metal powder or sheet: such as gold, silver, copper, iron and aluminum; (2) non-metallic and conductive particles: such as carbon black and carbon nanometer-tubes (CNTs); (3) surface-plated metal polymer particles; and (4) special carbon fibers. When the conductive particles reach the critical value, the properties thereof are different from those of the original material, the conductivity increases rapidly, and the polymer will transform from an insulator into a conductor. This phenomenon is called conductive seepage phenomenon, and the critical value of the volume mixed with conductive particles is called percolation thresholds.

Graphene is made from a monomolecular layer of graphite, is a honeycomb crystal lattice plane film composed of carbon atoms arranged by sp2 hybrid orbit, and is a two-dimensional nanometer material having only one carbon atom thickness. At present time, graphene is the world's thinnest (only one carbon atom thickness) and the hardest nanometer material, and it is almost completely transparent. Among them, graphene has three kinds of allotropes, such as zero dimensional Fullerence, one dimension carbon nanometer tubes and two-dimensional graphene.

Graphene is a honeycomb crystal lattice plane film composed of carbon atoms arranged by sp2 hybrid orbit, and is a two-dimensional material having only one carbon atom thickness. In the experiment, the graphene can be separated from the graphite, and it is confirmed that it can exist alone. Graphene is named from English graphite (graphite) +-ene (end of olefins). Graphene is considered to be a planar polycyclic aromatic hydrocarbon atom crystal. The structure of the graphene is very stable, and the carbon-carbon bond thereof is only 1.42 Å. The connection between the carbon atoms inside of the graphene is very flexible. When the external force is applied to the graphene, the surfaces of the carbon atoms are bent and deformed, so that the carbon atoms need not be rearranged to accommodate the external forces, thus maintaining the structural stability. This stable lattice structure gives the graphene excellent thermal conductivity. In addition, when the electrons in the graphene move in the track, they do not scatter due to lattice defects or the introduction of foreign atoms. As the inter-atomic force is very strong, there is very little interference at graphene internal electrons at room temperature even if the surrounding carbon atoms are crashed.

Graphene is currently not only the world's thinnest, but also the hardest nanometer-material, and it is almost completely transparent, only absorbing 2.3% of the light; its thermal conductivity is up to 5300 W/m·K, higher than the carbon nanometer-tubes and diamond; it has an electron mobility of more than 15,000 cm2/V·s, higher than that of carbon nanometer-tubes or mono crystalline silicon, and its resistivity is only about 10-6 Ω·cm, lower than copper or silver. Because it has a very low resistivity and a very fast electron movement, it is expected to be used to develop a new generation of electronic components or transistors that are thinner and faster in conductivity. Since graphene is essentially a kind of transparent and good conductor, it is also suitable for making a transparent touch screen, a light board, and even solar cells.

The main preparation methods of graphene are: a chemical vapor deposition (CVD) method, a mechanical exfoliation method, an organic molecular dispersion method, an ion intercalation method, a solvothermal and reduced grapheme oxide method, etc. The chemical vapor deposition method is a kind of thin film growth method which forms a graphene film on the surface of the substrate by a chemical reaction which the energy activates on the gas reaction precursor. Kim et al. generate two-dimensional graphene film by decomposition of CH4, reduction of CO. However, the aforementioned process is not mature, and the cost is high, thereby limiting the application in scale. A mechanical stripping method applies an ion beam on the surface of the material, and the graphene is produced by stripping off the surface of the material by a mechanic force. The monolayer graphene can be obtained from the highly oriented pyrolytic graphite by mechanical stripping. However, due to the complexity of the process, the yield of graphene is low, which cannot meet the industrial demand, thereby to some extent limiting the production in scale. The organic molecular dispersion method is a method to obtain graphene by dispersing the graphite in the organic solvent. The graphite can be successfully produced by ultrasonic dispersion in the organic solvent. Although this method has the advantages of less graphene defects, the concentration is not high. The ion intercalation method is firstly to produce the graphite intercalation compounds, and then disperse the organic compounds in the organic solvent to obtain Graphene. Penicoud et al. produce an alkali metal graphite interlayer compound and disperses it in N-methylpyrrolidone (NMP) to obtain a graphene dispersion liquid. By this way, the dispersion degree of the graphene is lower. The solvent thermal method is performed by adding the reactants to the solvent. By using the ability of a solvent to dissolve the vast majority of the material at a temperature higher than the critical temperature and the critical pressure, it can cause the reaction that can not occur under normal conditions to be carried out or accelerated at a lower temperature in a high pressure furnace. By using the ethanol and the metal sodium micro reactants, graphene is produced with the yield of grains level. Because this method is recently developed, and many theoretical and technical problems still can be not solved, it needs to be further explored.

An oxidation reduction method is produced by first manufacturing a graphene oxide, and then reducing it to prepare graphene by using reducing agents. This method has advantages of low cost and high yield rate, and is one of the best ways for the future mass production of graphene. The commonly used reducing agents are hydrazine, sodium borohydride, p-phenylenediamine, etc. Hydrazine has advantages of strong reducibility, low price, etc., and is currently widely used in the reducing process. Stankovich et al. studied the modification and reduction of graphene oxide. Firstly, the oxidized graphite is dispersed in water and reduced with hydrazine to obtain graphene. As oxygen contained in the surface of the graphene is decreased, the surface potential of the graphene is decreased, resulting in poor dispersibility of the graphene in the solvent and the occurrence of irreversible reunion phenomenon. In order to avoid agglomeration in the reduction process when the surface of the graphene is enclosed with a polymer, the graphene which is enclosed by using polystyrene sulfonate is used to form a colloidal dispersion. However, the addition of polymer dispersants affects the physical properties of graphene, thereby limiting its application in many ways. Li et al., on the basis of this study, by the method of using pure hydrazine to reduce the graphene oxide in the absence of any chemical stabilizer and by adding ammonia to change the pH to control the electrostatic repulsion between layers, produce stable graphene dispersion-fluid in the water phase.

The graphite oxide can be obtained by using nitric acid and potassium chloride. By using a mixture of sodium nitrate, potassium permanganate and concentrated sulfuric acid, it has also been found that oxidized graphite can be obtained more efficiently. A further improved method can peel off a lot of graphene oxide of a single atom layer. Since the base surface of the oxidized graphite has been bonded to a large number of oxidizing functional groups, the oxidizing groups act in a hydrophilic manner so that the uppermost layer of graphene peels off due to the van der Waals force between the graphite layers. In addition, the graphite block exposed to the new graphite base surface also has a hydrophilic surface. With such a mechanism, graphene oxide can be peeled off from the oxidized graphite block, like stripping onions layer by layer. However, according to this typical synthetic method, the size of the obtained graphene oxide is not large, and thus this typical synthetic method limits the practical application.

The step of producing the oxidized graphite block includes the help of ultrasonic vibration to acidify, which can help the subsequent stripping step, thereby obtaining a large area of graphene oxide. In addition, by adjusting the time of said vibration, different sizes of graphene sheet can be obtained. The maximum size of the graphene sheet obtained by this method can reach about 3 mm, which is the largest size in the literature. In addition to being able to be dispersed in water, the graphene oxide can be dispersed in a variety of customary solvents (SDS, DMF and NMP, etc.), thus providing a variety of subsequent applications.

In accordance with an embodiment of the present disclosure, the present invention discloses a sensing material for sensing a physiological parameter. The sensing material includes a carbon black material, a graphene material and a glue material, wherein the carbon black material, the graphene material and the glue material are mixed together based on a specific weight ratio.

In accordance with a further embodiment of the present disclosure, the present invention discloses a method for producing a sensing material unit for sensing a physiological parameter. The method includes the following steps. A wearable object is provided. A carbon black material, a graphene material and a glue material are provided with a specific weight ratio thereamong. The carbon black material, the graphene material and the glue material are mixed to form a mixed colloid. The mixed colloid are coated onto the wearable object, the mixed colloid are dried, and the coating and drying steps are repeated until appropriate coating layers are formed so that the mixed colloid and the wearable object form a to-be-manufactured treated wearable object. And, the to-be-manufactured treated wearable object is baked to form the sensing material unit being in a nano scale and having an electrical parameter.

In accordance with a further embodiment of the present disclosure, the present invention discloses a unit for sensing a physiological parameter from an organism. The unit includes a nano carbon layer and a protective layer. The nano carbon layer transmits a physiological signal in response to the physiological parameter. The protective layer protects the nano carbon layer.

The above embodiments and advantages of the present invention will become more readily apparent to those ordinarily skilled in the art after reviewing the following detailed descriptions and accompanying drawings.

### BRIEF DESCRIPTION OF THE DRAWINGS

FIG. 1 shows a conductive material of an inner layer of a smart cloth in the prior art;

FIG. 2 shows a sensing material being touchable with an organism for sensing a physiological parameter according to the preferred embodiment of the present disclosure;

FIG. 3 shows respective volume resistivity of different materials according to the preferred embodiment of the present disclosure;

FIG. 4 shows a flow chart of manufacturing a unit for sensing a physiological parameter from an organism according to the preferred embodiment of the present disclosure;

FIG. 5 shows a unit made by using a nano-carbon mixture material for sensing a physiological parameter from an organism according to the preferred embodiment of the present disclosure;

FIG. 6 shows a method for manufacturing a unit for sensing a physiological parameter from an organism according to the preferred embodiment of the present disclosure;

FIG. 7(a) shows a sensor integrated into a unit for sensing a physiological parameter from an organism according to the preferred embodiment of the present disclosure;

FIG. 7(b) shows an average electrocardiogram (ECG) by using a metal serving as a sensing material according to the preferred embodiment of the present disclosure;

FIG. 7(c) shows an average electrocardiogram (ECG) by using a nano-carbon mixture film serving as a sensing material according to the preferred embodiment of the present disclosure;

FIG. 8 shows a resistance status depending on washing times according to the preferred embodiment of the present disclosure;

FIG. 9(a) shows an ECG signal of the carbon material in a static condition;

FIG. 9(b) shows an ECG signal of the carbon-graphene material in the static condition;

FIG. 10(a) shows an entire ECG of the electrode of the carbon-graphene material according to the preferred embodiment of the present disclosure;

FIG. 10(b) shows heart rates in the unit of beat/second according to the preferred embodiment of the present disclosure;

FIG. 10(c) shows an ECG signal when the man is still during 0 to 60 seconds;

FIG. 10(d) shows an ECG signal while the man is walking during 120 to 180 seconds; and

FIG. 10(e) shows an ECG signal while the man is jogging and then running during 180 to 250 seconds.

### DETAILED DESCRIPTION OF THE PREFERRED EMBODIMENTS

Please refer to all Figs. of the present invention when reading the following detailed description, wherein all Figs. of the present invention demonstrate different embodiments of the present invention by showing examples, and help the skilled person in the art to understand how to implement the present invention. The present examples provide sufficient embodiments to demonstrate the spirit of the present invention, each embodiment does not conflict with the others, and new embodiments can be implemented through an arbitrary combination thereof, i.e., the present invention is not restricted to the embodiments disclosed in the present specification.

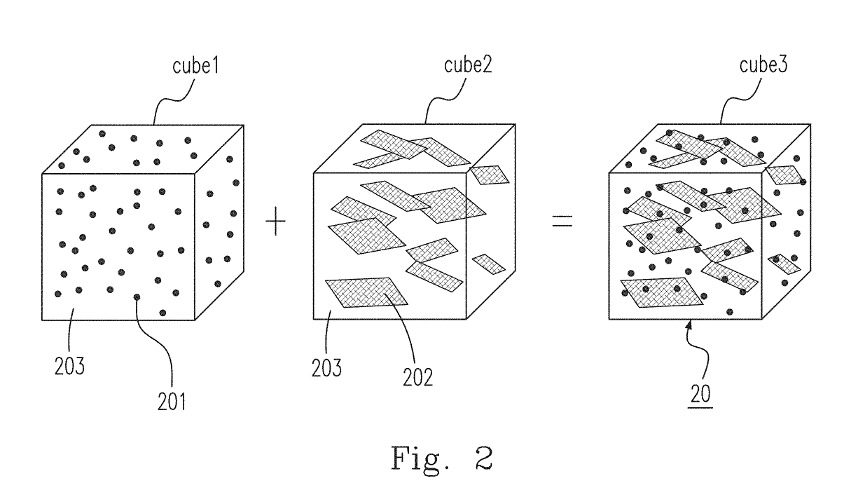

Please refer to FIG. 2, which shows a sensing material being touchable with an organism for sensing a physiological parameter according to the preferred embodiment of the present disclosure. The sensing material 20 for sensing a physiological parameter includes a carbon black material 201, a graphene material 202 and a glue material 203, wherein the carbon black material 201, the graphene material 202 and the glue material 203 are mixed together based on a specific weight ratio.

FIG. 2 also shows materials mixed together in a micro scale view according to the preferred embodiment of the present disclosure. A cube cube1 includes the carbon black material 201 with grain shape and the glue material 203 entirely filling in the cube cube1. The carbon black material 201 is a non-metal, and is also an electrical-conductive material. In FIG. 2, it can be seen that the carbon black material 201 is uniformly distributed in the glue material 203. A cube cube2 includes the graphene material 202 having a film-like shape and the glue material 203. The grapheme material 202 is also an electrical-conductive material among the non-metal materials, and it is distributed in the glue material 203 by an irregular stack of layers. A conductivity of a single flake-like or film-like grapheme in the graphene material 202 is superior; however, a yield rate should be taken into consideration while obtaining the single flake-like or film-like graphene by mass-production in the practical application. In fact, the yield rate is low, and only a stack of multiple layers can be obtained in most cases. Because there is a little spare space between the stack of layers, causing a relatively high resistance characteristic, and thus the graphene material 202 does not always meet the requirement of a good conductor.

In the preferred embodiment of the present disclosure, the carbon black material 201 is added to fill the spare space between the stack of layers of the grapheme material 202, reducing the resistance thereof, so as to raise its conductivity. Referring to the cube cube3 in FIG. 2, the carbon black material 201 is uniformly distributed between the graphene material 202 and the glue material 203, and thus the sensing material 20 being wearable, electrical-conductive and non-metal can be formed. The carbon black material 201 and the graphene material 202 are mixed together to form a nano-carbon material, wherein the glue material 203 is added into the nano-carbon material, and it can improve stretching and flexibility of the nano-carbon material. These three materials mixed together in a specific range of weight ratio can form a nano carbon-mixture material, which has a good stretching, and can be applied to the conductive layer of the smart cloth to improve comfort while the smart clothing is being worn on the human body. This kind of material is different from the traditional fabric manufactured by with metal fiber, which lacks of flexibility by its nature, and thus decreases the comfort when worn. If there is a need to increase the comfort, it is time consuming and complex for a production process using a traditional material and a traditional production method, and does not meet the requirement of the cost benefit.

The carbon black material 201 is an allotrope of the carbon family. The carbon black material 201 adapted by the preferred embodiment of the present disclosure is Ketjen black of Bell Lab company, and it includes product types EC600JD and EC300J. The different product types have different characteristics that can be used to tell them apart. Please refer to the following Table 1.

TABLE 1

| | EC300J | EC600JD |
|---|---|---|
| Absortion rate | 360 | 495 |
| towards dibutyl | | |
| phthalate (DBP) | | |
| (cm³/100 g) | | |
| BET surface | 800 | 1270 |
| area (m²/g) | | |
| Volatiles (%) | 0.5 | 0.7 |
| pH | 9 | 9 |
| Ash content (%) | 0.05 | 0.10 |
| Primary particle | 39.5 | 34.0 |
| radius | | |

| | |
|---|---|
| Applications | Electro-conductive filler used in resin compounds, |
| | battery materials, colorant and toner |

In Table 1, it can be seen that the different carbon black material 201 has a different absorption rate towards dibutyl phthalate (DBP). In the preferred embodiment of the present disclosure, cm³/100 g is used as a unit, the absorption rate of EC600JD is higher than that of EC300J, and the BET surface area of EC600JD is also bigger than that of EC300J. The BET surface area refers to a total area in which the material has per unit mass, and m²/g is used as a unit in the preferred embodiment of the present disclosure. The primary particle radius of EC600JD is also smaller than that of EC300J, ash content of EC600JD is higher than that of EC300J, Volatility of EC600JD is higher than that of EC300J, and pH values are the same.

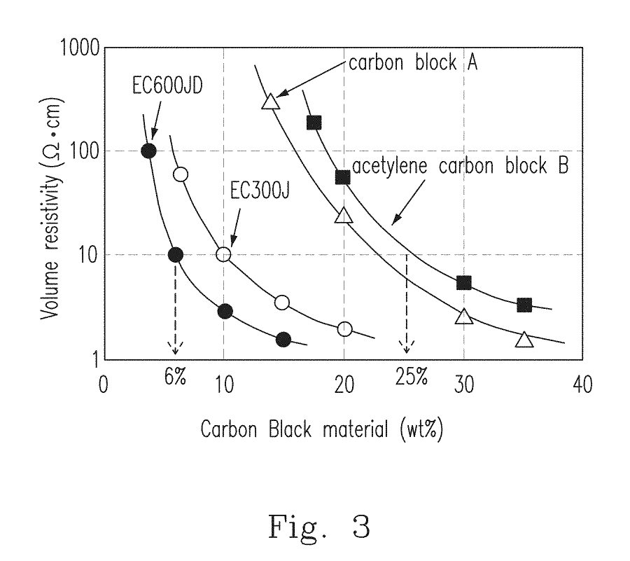

From another point of view, the value of the volume resistivity can be used to distinguish them from each other. The volume resistivity can be called a resistance coefficient, is an indicator that can measure impedance or conductivity of a material. Please refer to FIG. 3, which shows the respective volume resistivity of different materials according to the preferred embodiment of the present disclosure. The horizontal axis represents the weight percentage concentration of different kinds of carbon black material 201 in the resin including high density polyethylene (HDPE) for example, and wt % is used as a unit. The vertical axis represents respective volume resistivity of different kinds of carbon 201, and Ω·cm is used as a unit. In FIG. 3, the four curved lines represent the volume resistivity of carbon black A, acetylene carbon black B, EC600JD and EC300J respectively under different weight percentage concentration, and they are connected by hollow triangles, solid squares, solid circles and hollow circles respectively. In FIG. 3, it can be seen that the volume resistivity of the acetylene carbon black B is 10 Ω·cm, higher than that of the carbon black A at the same weight percentage concentration of 25%. It apparently shows that the conductivity of the carbon black A is superior to that of the acetylene carbon black B. Similarly, it can be seen that the volume resistivity of EC600JD is 10 Ω·cm, lower than that of EC300J at the same weight percentage concentration 6%. It apparently shows that the conductivity of EC600JD is not only superior to that of EC300J, but also superior to the other two materials because it shows that only a small amount of EC600JD is needed to reach the same satisfaction conductivity, which is superior to those of the other three kinds of carbon black A, B and EC300J.

The graphene material 202 in the preferred embodiment of the present disclosure includes product types PML, PMF and PHF of Taiwan Enerage Inc., and product types TPGnP001, TPGnP002 and TPGnP003 of Taiwan Textile Research Institute (TTRI). The differences between these product types of graphene are oxygen content, layers and thickness, wherein the different oxygen contents depend on their functional groups respectively. For examples, functional groups are the hydroxyl group (OH), the carboxyl group (COOH) and the like. Oxygen content is related to conductivity of the material. The less oxygen content it contains, the less impedance or resistivity between layers of the graphene material 202 it will have, and thus the conductivity is better. The following Table 2 shows the comparison of the characteristics between product types PML20 and P-LF10F of Taiwan Enerage Inc. for identifying two kinds of graphene material 202.

TABLE 2

| | PML20 | | P-LF10 | |
|---|---|---|---|---|
| Layer count | More stack of layers | | Fewer stack of layers | |
| Appearance | Gray fluffy powder | | Black fluffy powder | |
| Oxygen content | <3 | wt % | <3 | wt % |
| Tap density | ~0.025 | g/cm3 | ~0.001 | g/cm3 |
| Specific surface area | ~25 | m²/g | ~350 | m²/g |
| Average lateral size | ~25 | μm | ≤2 | μm |
| (X-Y plane) | | | | |
| Electrical conductivity | ≥19 | S/cm | ≥350 | S/cm |

Table 2 shows that types PML20 and P-LF10 are identified by layer count, appearance, oxygen content, tap density, specific surface area, average lateral size and electrical conductivity. Electrical conductivity is a reciprocal of resistivity. In Table 2, it can be seen that the electrical conductivity of the type PML20 is larger than 19 S/cm, the electrical conductivity of the type P-LF10 is larger than 350 S/cm, both oxygen contents are below 3 wt %. However, the stack of layers of the type PML20 is more than those of the type P-LF10, the more stacks of layers, the larger the impedance or resistance it has according to the aforementioned. Therefore, the electrical conductivity of the type PML20 is lower than that of the type P-LF10.

The following Table 3 shows the comparison of the characteristics among product types TPGnP001, TPGnP002 and TPGnP003 of TTRI for identifying three kinds of graphene material 202.

TABLE 3

| | | Base | | | Carbon | Water | Volume | Real | BET |
|---|---|---|---|---|---|---|---|---|---|
| | | surface | Thickness | Layer | Content | Absorption Rate | Density | Density | Surface Area |
| Product Type | | (μm) | (μm) | Count | (%) | (%) | (g/ml) | (g/ml) | (m2/g) |
| TTRI | TPGnP-001 | 10-15 | 0.003-0.005 | 6-10 | ≥99 | ≤2 | 0.03-0.1 | 25-50 | 2.25 |
| | TPGnP-002 | 5-10 | 0.003-0.005 | 6-10 | ≥99 | ≤2 | 0.03-0.1 | 25-50 | 2.25 |
| | TPGnP-003 | 3-5 | 0.003-0.005 | 6-10 | ≥99 | ≤2 | 0.03-0.1 | 25-50 | 2.25 |

In Table 3, the only difference among these three graphene materials 202 of the type TPGnP-001, TPGnP-002 and TPGnP-003 is in their base surfaces. The base surface will effect an interlacing degree of the graphene material 202 and a stack degree of the graphene material 202, an inner porosity, and so on. The type TPGnP-001 having a base surface of 10-15 μm has a higher interlacing degree and a higher stack degree, and has a denser lateral surface structure. However, the type TPGnP-003 having a base surface of 3-5 μm has a higher inner porosity. The interlacing degree, the stack degree and the inner porosity will not only effect the impedance of the material, but also the conductivity.

In the preferred embodiment of the present disclosure, the sensing material 20 for wearing and sensing the physiological parameter is mixed together by the carbon black material 201, the graphene material 202 and the glue material 203 by using a specific range of a weight ratio, rather than an arbitrary weight ratio. The carbon black material 201 can uniformly distribute and fill the space between layers of the graphene material 202, and the glue material 203 is also added thereinto to improve the flexibility and the stretching ability. Thus, it is not easy to achieve both conductivity and flexibility by using a traditional metal fabric and an ordinary skill.

The following Table 4 shows a mixture formula used to mix the carbon black material 201, the graphene material 202 and the glue material 203 in different weight percentage ratios.

TABLE 4

| | | | Resistance |
|---|---|---|---|
| | | Resistance | measured by |
| | Abbreviation | measured by | four point |
| Preferred | of Electrical | Multimeter | probe method |
| embodiment | colloid | (KΩ) | (Ω) |
| Selection of | | | |
| Graphene | | | |
| material PML | | | |
| Embodiment 1 | CB-PML-1:0 | 3.6 | 540 |
| Embodiment 2 | CB-PML-0:1 | N/A | N/A |
| Embodiment 3 | CB-PML-1:1 | 4.1 | 630 |
| Embodiment 4 | CB-PML-2:1 | 2.60 | 480 |
| Embodiment 5 | CB-PML-1:2 | 3.05 | 510 |
| | | Resistance | |
| | Abbreviation | measured by | |
| | of Electrical | Multimeter | [symbol] |
| Embodiment | colloid | (KΩ) | (Ω) |
| Selection of | | | |
| Graphene | | | |
| material TPGn001 | | | |
| Embodiment 6 | CB- TPGnP001-0:1 | N/A | N/A |
| Embodiment 7 | CB- TPGnP001-1:1 | 4.8 | 720 |
| Embodiment 8 | CB- TPGnP001-2:1 | 2.95 | 630 |
| Embodiment 9 | CB- TPGnP001-1:2 | 3.6 | 590 |

In Table 4, N/A in the Embodiments 2 and 4 show that the resistance is too large to measure. The carbon black material 201 in Embodiments 1-9 is abbreviated to “CB”, the graphene material 202 has two kinds of material including types PML and TPGnP001, and the carbon black material 201 and the graphene material 202 are mixed with the glue material 203 to form an electrical-conductive colloid in different weight ratios. The abbreviation of the electrical-conductive colloid mixed by different ingredients and ratios are represented by CB-PML-weight ratio or CB-TPGnP001-weight ratio. The carbon black material 201 and the graphene material 202 are mixed to form a nano carbon material. In Table 4, after the nano carbon material is mixed with the glue material 203, its weight percentage concentration is fixed at 5%. The glue material 203 includes at least one selected from the group consisting of polyurethane (PU), water polyurethane (WPU), thermoplastic PU, Silicone, Epoxy, rubber and plastisol. The carbon black material 201 includes at least one of product types EC600JD and EC300J. The graphene material 202 includes at least one selected from the group consisting of product types PML, PMF, PHF, TPGnP001, TPGnP002 and TPGnP003. In Table 4, it shows only one kind of ingredient of the graphene material 202 to mix with other material. Alternatively, more than one ingredient of the graphene material 202 can be mixed with the carbon black material 201 and the glue material 203. In addition, more than one ingredient of the carbon black material 201 can be mixed with other material. The nano carbon material may include a nano carbon tube material.

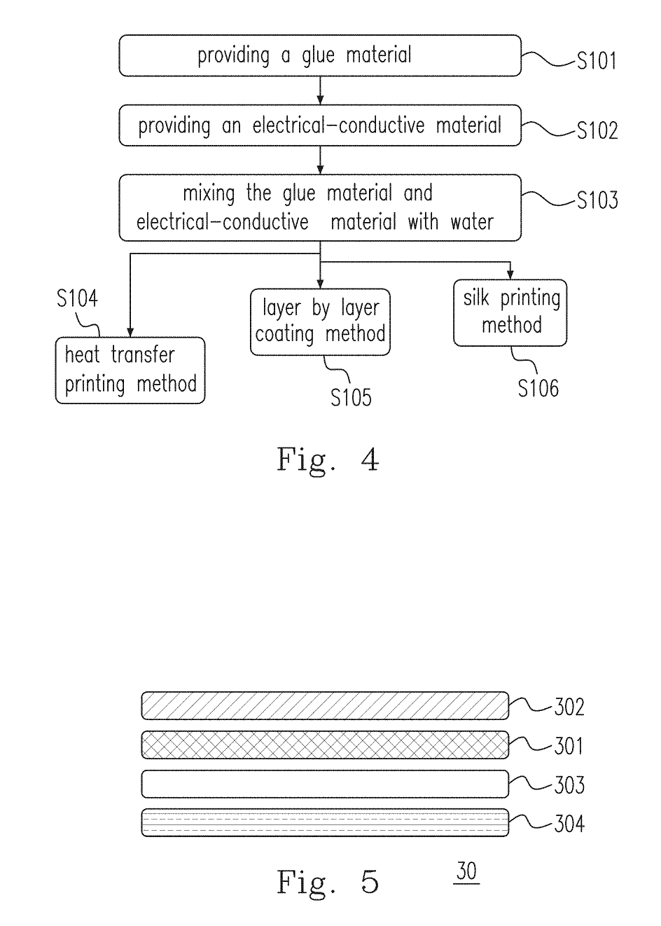

Please refer to FIG. 4, which shows a flow chart for manufacturing a unit for sensing a physiological parameter from an organism according to the preferred embodiment of the present disclosure. The preferred embodiment of the present disclosure uses the nano carbon material to manufacture an electrical-conductive colloid, and distributes the electrical-conductive colloid to a variety of resin glue by a drum grinding machine, a compound mulling machine or a mechanical blade stirring them. For example, the electrical-conductive colloid can be the carbon material 201, or a mixture material mixed by the carbon black material 201 and the graphene material 202. For example, the glue material 203 can be the PU, the silicone, the Epoxy and the rubber. Finally, a film is formed on the surface of various functional elastic sheets by means of a treatment method such as a scraper brush.

In FIG. 4, the flow chart of manufacturing the unit includes steps of S101-S106. Step S101: providing a glue material. Step S102: providing an electrical-conductive material. Step S103: mixing the glue material and the electrical-conductive material with water to form an electrical-conductive colloid. The electrical-conductive colloid can be attached on the sensing material 20 for sensing a physiological parameter from an organism by the heat transfer printing method in Step S104, layer by layer coating method in Step S105 or silk printing method in Step S106.

Please refer to FIG. 5, which shows a unit 30 made by using a nano-carbon mixture material for sensing a physiological parameter from an organism according to the preferred embodiment of the present disclosure. The unit 30 includes a nano carbon layer 301, a protective layer 302, a base layer 303 and a fabric layer 304. The protective layer 302 uses a relatively high impedance or resistance material as an anti-scraped layer. The nano carbon layer 301 has a relatively low impedance or resistance to serve as the electrical-conductive layer. The base layer 303 is used to combine with the fabric layer 304 in order to prevent the nano carbon layer 301 from falling off.

The unit 30 for sensing a physiological parameter from an organism includes at least one of a fabric and a wearable unit. The fabric includes a smart fabric including at least one of a smart sleeping quilt, a smart rain coat and a smart umbrella. The wearable unit includes at least one selected from the group consisting of a smart wristband, a smart buckle, a smart watch, a smart cloth, a smart earphone, a smart glass, a smart diaper, a smart banding, a smart heart rate meter, a smart heart rhythm meter and a smart leather-made product. The preferred embodiment in the present disclosure uses the electrical-conductive colloid to coat on the fabric to form films, and wherein the electrical-conductive colloid is made by the nano carbon mixture material and is to be a sensor or a transmitter.

In addition, the electrical-conductive colloid made by the nano carbon mixture material can be applied to many units 30, and there are many units 30 that can be applied to a one single smart cloth in order to sense a physiological parameter including at least one selected from the group consisting of a heart rate, a heart rhythm, a breath rate, a blood pressure, a pulse, an ECG parameter, a pace count, a strength of activity and a calorie consumption.

Please return to FIG. 4, the manufacturing process flow of a variety of contents and weight ratios are described as follows.

Preferred embodiment 1: CB-Graphene-WPU filler is mixed together by the weight ratio 1:0. Firstly, taking 5 g EC600JD, adding it into a plastic bottles having a volume of 500 Ml; and add 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, putting it into the oven at a temperature of 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 540 ohms, and the resistance value obtained by measuring the two points at an arbitrary distance of less than 5 cm is 3.6 kΩ by using the multimeter. In all embodiments, the four-point probe meter is used to measure the surface resistance coefficient in a material, while the multimeter is mainly used to measure the overall resistance coefficient of the material. It is often possible to detect whether the material and the circuit are stable during the manufacturing process by measuring these parameters of resistances. One of the key factors of affecting product yield rate is the stability of process parameters.

Preferred embodiment 2: CB-Graphene-WPU filler is mixed together by the weight ratio 0:1. Firstly, taking 5 g PML, adding it into a plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto its surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm. Because there is no carbon black material 201 in the Embodiment 2, only graphene material 202 is contained therein, according to the aforementioned, there will be holes between a stack of layers in the graphene material 202, it will reduce the conductivity of the nano carbon mixture material, and thus the resistance is too large to measure. The amount of water can be added properly. For example, the amount of the water added to the glue material 203 can be the same as that of the glue material 203, so that the mixed electrical-conductive colloid has a stickability dependent on the volume of the water.

Preferred embodiment 3: CB-Graphene-WPU filler is mixed together by the weight ratio 1:1. Firstly, taking 2.5 g EC600JD and 2.5 g PML, adding them into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at a temperature of 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 630 ohms, and the resistance value obtained by measuring the two points at an arbitrary distance of less than 5 cm is 4.1 kΩ by using the multimeter.

Preferred embodiment 4: CB-Graphene-WPU filler is mixed together by the weight ratio 2:1. Firstly, taking 3.33 g EC600JD and 1.67 g PML, adding them into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 480 ohms, and the resistance value obtained by measuring the two points at an arbitrary distance of less than 5 cm is 2.6 kΩ by using the multimeter. The conductivity of the unit 30 in Embodiment 4 is better than that of Embodiment 3 because the weight of carbon black material 201 in Embodiment 4 is twice the weight as in Embodiment 3 and it fills the space between the stack of layers in the graphene material 202, and thus increases the conductivity of the nano carbon mixture material.

Preferred embodiment 5: CB-Graphene-WPU filler is mixed together by the weight ratio 1:2. Firstly, taking 1.67 g EC600JD and 3.33 g PML, adding them into a plastic bottles having a volume of 500 Ml, and add 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 510 ohms, and the resistance value obtained by measuring the two points at an arbitrary distance of less than 5 cm is 3.05 kΩ by using the multimeter. The conductivity of the unit 30 in Embodiment 5 is better than that of Embodiment 3 because the weight of the graphene material 202 in Embodiment 5 is twice that in Embodiment 3 under the situation of the same weight of the carbon black material 201, and thus increases an extended ability of the single layer in the graphene material 201, so as to increase the conductivity thereof. However, its conductivity will decrease if too much graphene material 202 is added, the conductivity thereof will decrease because the stack of layers will increase.

Preferred embodiment 6: CB-Graphene-WPU filler is mixed together by the weight ratio 0:1. Firstly, taking 5 g TPGn001, adding it into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm. Because there is no carbon black material 201 in the Embodiment 6, only graphene material 202 is contained therein, according to the aforementioned, so there will be holes between a stack of layers in the graphene material 202. It will reduce the conductivity of the nano carbon mixture material, and thus the resistance is too large to measure.

Preferred embodiment 7: CB-Graphene-WPU filler is mixed together by the weight ratio 1:1. Firstly, taking 2.5 g EC600JD and 2.5 g TPGn001, adding them into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at a temperature of 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 720 ohms, and the resistance value obtained by measuring the two points at an arbitrary distance of less than 5 cm is 4.8 kΩ by using the multimeter.

Preferred embodiment 8: CB-Graphene-WPU filler is mixed together by the weight ratio 2:1. Firstly, taking 3.33 g EC600JD and 1.67 g TPGn001, adding them into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 630 ohms, and the resistance value obtained by measuring the two points at an arbitrary distance of less than 5 cm is 2.9 kΩ by using the multimeter.

Preferred embodiment 9: CB-Graphene-WPU filler is mixed together by the weight ratio 1:2. Firstly, taking 1.67 g EC600JD and 3.33 g TPGn001, adding them into plastic bottles having a volume of 500 Ml, and adding 95 mL WPU to mix uniformly by using a mechanical mixer stirring them uniformly. In addition, 95 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm to be 590 ohms, and the resistance value obtained by measuring the two points at an arbitrary distance of less than 5 cm is 3.6 kΩ by using the multimeter.

Please refer to the following Table 5, which shows a mixed formulation of the carbon black material 201, the graphene material 202 and the glue material 203 in the preferred embodiment according to the present disclosure.

TABLE 5

| | | | Four | Four point |
|---|---|---|---|---|
| | | | point | probe value |
| | Abbreviation | | probe | (washed by |
| Preferred | of Electrical | Multimeter | value | water) |
| embodiment | colloid | (KΩ) | (Ω) | (Ω) |
| Graphene:PML | | | | |
| 10 | (carbon material | N/A | N/A | N/A |
| | ratio CB-PML-1:2) | | | |
| | 4 wt % in PU | | | |
| 11 | (carbon material | 2 | 180 | — |
| | ratio CB-PML-1:2) | | | |
| | 6 wt % in PU | | | |
| 12 | (carbon material | 1.6 | 150 | 250 |
| | ratio CB-PML-1:2) | | | |
| | 11 wt % in PU | | | |
| 13 | (carbon material | — | — | — |
| | ratio CB-PML-1:2) | | | |
| | 7 wt % in PU | | | |
| 14 | (carbon material | — | — | — |
| | ratio CB-PML-1:2) | | | |
| | 10 wt % in PU | | | |

The difference between Tables 4 and 5 is that the weight percentage concentration wt % of the nano carbon material (including the carbon black material 201 and the graphene material 202) in the glue material 203 gradually increases. N/A in Table meants that the resistance is too large to measure, and the dash line indicates that the resistance approaches zero.

Preferred embodiment 10: the carbon material ratio of CB-PML in PU is 1:2, and the weight percentage concentration of CB-PML in PU is 4 wt %. Firstly, taking 2.67 g EC600JD and 1.33 g PML, adding them into plastic bottles having a volume of 500 Ml, and adding 95 mL WPU to mix uniformly by using a mechanical mixer stirring them uniformly. In addition, 96 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism is obtained. Finally, a four-point probe is used to measure a surface resistance on a fabric of 10×10 cm. Because the weight percentage of the nano carbon material is small, the characteristic of conductivity cannot be detected, and the impedance or the resistance is too large to measure.

Preferred embodiment 11: the carbon material ratio of CB-PML in PU is 1:2, and the weight percentage concentration of CB-PML in PU is 6 wt %. Firstly, taking 4 g EC600JD and 2 g PML, adding them into plastic bottles having a volume of 500 Ml, and adding 94 mL WPU to mix uniformly by using a mechanical mixer stirring them uniformly. In addition, 94 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism will be obtained. Finally, a four-point probe is used to measure a surface resistance on a fabric of 10×10 cm to be 180 ohms, and the resistance value obtained by measuring the two points at an arbitrary distance of less than 5 cm is 2 kΩ by using the multimeter.

Preferred embodiment 12: the carbon material ratio of CB-PML in PU is 1:2, and the weight percentage concentration of CB-PML in PU is 11 wt %. Firstly, taking 4.67 g EC600JD and 2.33 g PML, adding them into a plastic bottles having a volume of 500 Ml, and adding 93 mL WPU to mix uniformly by using a mechanical mixer stirring them uniformly. In addition, 93 mL of water is slowly added to the colloid, and the mixture is homogenized and stirred for 15 minutes at 25° C. using a three-roll kneader. The mixed electrical-conductive colloid is coated onto the flexible fiber cloth by using a scraper brush, and is blown dry with a heat air gun. Repeatedly coating three layers to five layers onto the surface of the flexible fiber cloth, and putting it into the oven at temperature 130° C. for reaction for 5 minutes; and then the unit 30 made by the nano carbon mixture material for sensing the physiological parameter from the organism will be obtained. Then, a four-point probe is used to measure a surface resistance on a fabric of 10×10 cm to be 150 ohms, and the resistance value obtained by measuring the two points at an arbitrary distance of less than 5 cm is 1.6 kΩ by using the multimeter. Finally, two pieces of the unit 30 are cut to attach onto the cloth as an electrode layer of the smart cloth.

Each of the preferred embodiments in the present disclosure only shows a preferred weight ratio, these materials can be mixed by other weight ratios, those skilled in the art can implement the preferred embodiments without taking undue experiments by mixing these materials in other ratios, which is still in the scope of the present disclosure. For examples, the weight ratio of the carbon black material 201 and the graphene material 202 can be 0˜2:2˜0, i.e., when the carbon black material 201 decreases from the ratio 2, the ratio of the graphene material 202 increases from zero ratio, but both total ratios are still 2. Similarly, the weight percentage concentration of the carbon black material 201 and the graphene material 203 in the glue material 203 can be 5˜11 wt %. For examples, when the weight percentage concentration of the carbon black material 201 and the graphene material 202 increases from 5 g, the weight of the glue material 203 decreases from 95 g, but the total weight of the material is still 100 g.

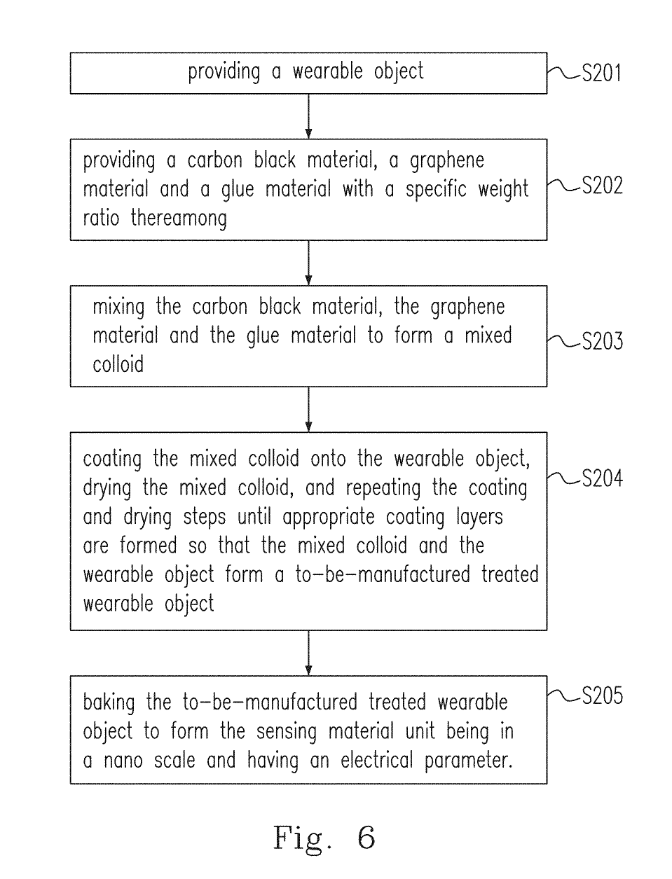

Please refer to FIG. 6, which shows a method for producing a sensing material unit for sensing a physiological parameter, the method includes the following steps. Step S201: providing a wearable object. Step S202: providing a carbon black material, a graphene material and a glue material with a specific weight ratio thereamong. Step S203: mixing the carbon black material, the graphene material and the glue material to form a mixed colloid. Step S204: coating the mixed colloid onto the wearable object, drying the mixed colloid, and repeating the coating and drying steps until appropriate coating layers are formed so that the mixed colloid and the wearable object form a to-be-manufactured treated wearable object. Step S205: baking the to-be-manufactured treated wearable object to form the sensing material unit being in a nano scale and having an electrical parameter.

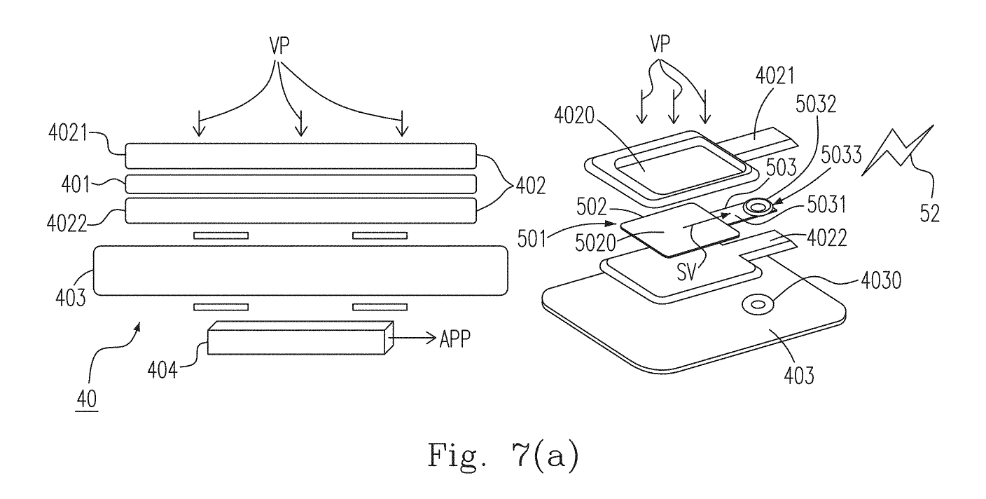

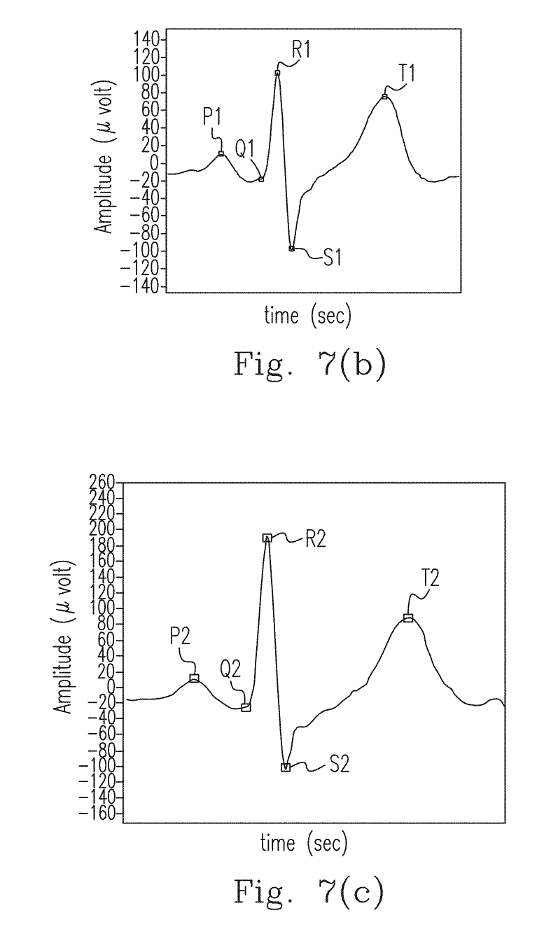

Please refer to FIG. 7(a), which shows a unit 40 for sensing a physiological parameter VP from an organism. The unit 40 includes a nano carbon layer 401 and a protective layer 402. The nano carbon layer 401 transmits the physiological signal VP in response to the physiological parameter. The protective layer 402 protects the nano carbon layer 401, wherein the nano carbon layer 401 is a sensor unit 501. The nano carbon layer 401 includes a nano carbon-mixture material (not shown), a sensing portion 502 and a transmission portion 503 coupled to the sensing portion 502. The protective layer 402 has at least one characteristic of water-isolation and electricity-isolation to prevent the nano carbon layer 401 from being destroyed, and includes a first protective layer 4021 and a second protective layer 4022. One of the first protective layer 4021 and the second protective layer 4022 has an opening structure 4020. In one preferred embodiment according to the present disclosure, the opening structure 4020 of the first protective layer 4021 is used to touch the skin. The sensing portion 502 senses the physiological parameter VP through the opening structure 4020. The physiological parameter VP includes at least one selected from the group consisting of a heart rate, a heart rhythm, a breath rate, a blood pressure, a pulse, an electrocardiogram (ECG) parameter, a pace count, a strength of activity and a calorie consumption. The transmission portion 503 transmits the physiological signal SV. The sensing portion 502 includes an electrode 5020. The transmission portion 503 includes at least one of a wire 5031 and a wireless transceiver 5032 including an RFID transceiver. The electrode 5020 and the wire 5031 can be made by the nano carbon mixture material. The wireless transceiver 5032 can be a coil made by the nano carbon mixture material in order to transmit a wireless signal 52. In one preferred embedment, the nano carbon mixture material can make a buckle for fixing an external wireless module, which is another module separated from the sensor unit 501 and can be coupled to an external buckle 4030, and is also detachable. The unit 40 further includes a cloth body 403 and a processing unit 404. The external buckle 4030 on the cloth body 403 can be electrically connected to the internal buckle 5033 made by the nano carbon mixture material, so as to transmit the physiological signal SV to the processing unit 404 to process. The cloth body 403 encloses the protective layer 402. The processing unit 404 is disposed on the cloth body 403, receives the physiological signal SV from the sensor unit 501, and processes the physiological parameter VP to display it in a graphic form according to an application program. The physiological signal SV may be transmitted in a wired manner between the processing unit 404 and the transmission unit 503, and is not limited thereto.

Please refer to FIG. 7(b), which shows an average electrocardiogram (ECG) by using a metal serving as a sensing material according to the preferred embodiment of the present disclosure. Please refer to FIG. 7(c), which shows an average electrocardiogram (ECG) by using a nano-carbon mixture film serving as a sensing material according to the preferred embodiment of the present disclosure. The horizontal axis represents the time in seconds, and the vertical axis represents the amplitude of the sensed voltage in micro volts. Q1 and Q2, R1 and R2, S1 and S2 are QRS waves, which represent the depolarization of the left and right ventricles. T1 and T2 are T waves, which represent the depolarization of the ventricular. In FIG. 7(b) and FIG. 7(c), the waveforms of P1 and P2 are similar, the waveforms of Q1, R1 and S1 are similar to those of Q2, R2 and S2, and the waveforms of T1 and T2 are similar. The average ECG measured by the sensor unit 501 made by the nano carbon mixture film can achieve the efficacy of the average ECG measured by a sensor made by the metal material, and the nano carbon mixture film having the elasticity serves as the sensing material to increase the comfort while wearing it. In addition, the process of production is simple, and the yield rate is high.

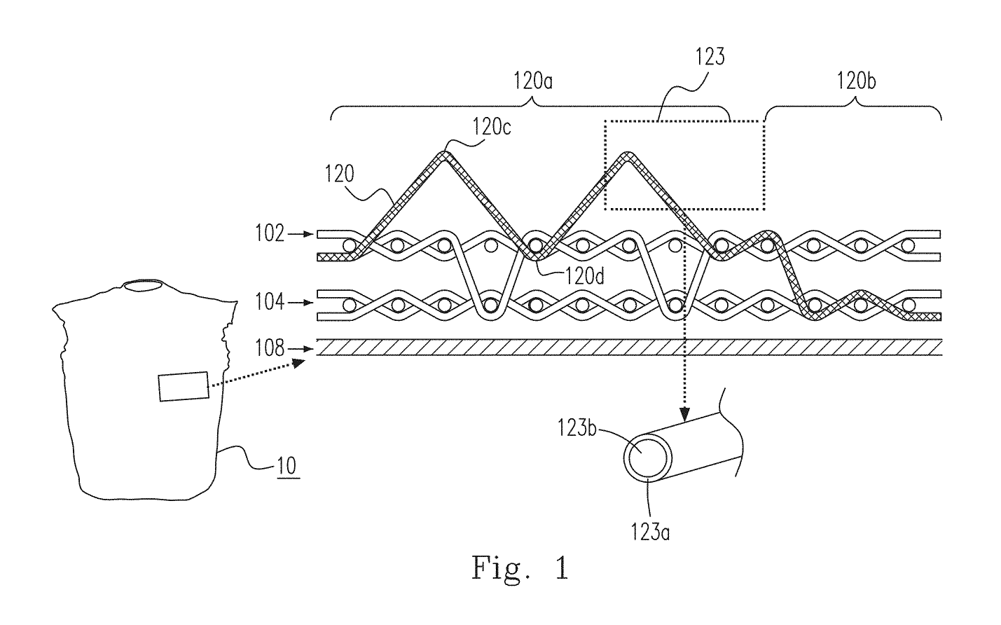

In FIG. 7(a), the unit 40 for sensing a physiological parameter VP from an organism is totally different from the fabric 10 in FIG. 1. The fiber layer 123b in the fabric 10 is plated with a metal plating layer 123a to form the first and second layers of fabric 102 and 104, which is interwoven with each other to form conductive yarns 120. On the contrary, the unit 40 for sensing a physiological parameter VP from an organism in the present invention has distinct layers. For examples, the nano carbon layer is separate from the layers of the cloth body 403 without mixing the two materials to form the conductive yarn 120, and therefore their structures are completely different.

The preferred embodiment of the present invention incorporates a nano carbon mixture film serving as a sensing material, and the nano carbon mixture film is adhered to the fabric by a coating method, a heat transfer printing method, or a silk printing method. The unit 40 for sensing a physiological parameter VP from an organism can be washed for a specified number of times, for example at least 20 times, and then dried. After the unit 40 is dried, a four-point probe method is used to measure the resistance, and the measured resistance value meets the requirements of smart clothing.

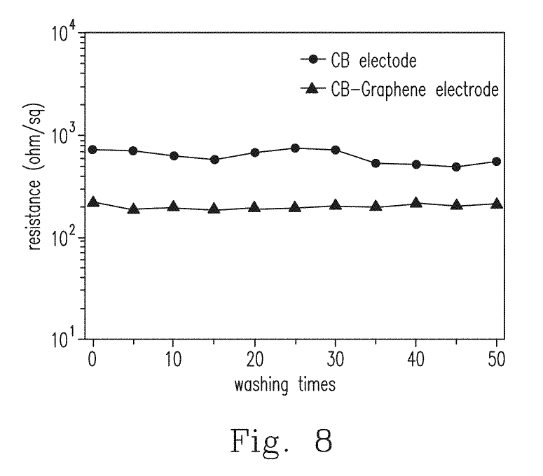

Please refer to FIG. 8, which shows a resistance status depending on washing times according to the preferred embodiment of the present disclosure. The horizontal axis in FIG. 8 represents washing times after the electrode 5020 is bound to the cloth body 403 as shown in FIG. 7(a), and the electrode 5020 includes the carbon material 201 or the carbon-graphene material as referred to in cube3 in FIG. 2. The vertical axis in FIG. 8 represents resistance per unit square (for example, cm2) of the electrode 5020; and the resistances of the carbon material 201 and the carbon-graphene material (201+202) are represented by a triangle shape and a circle respectively. In FIG. 8, it can be seen that the graphene can be used to reduce the resistance after it is added into the carbon material 201 because its high area ratio characteristic can contribute to conductivity of the carbon material 201. In addition, when the carbon material 201 or the carbon-graphene material is employed, each resistance of the carbon electrode and the carbon-graphene electrode stays at the same condition even after washing fifty times. For example, the carbon material's resistance is about 580-730 Ω/sq, and the carbon-graphene material's resistance is about 160-200 Ω/sq.

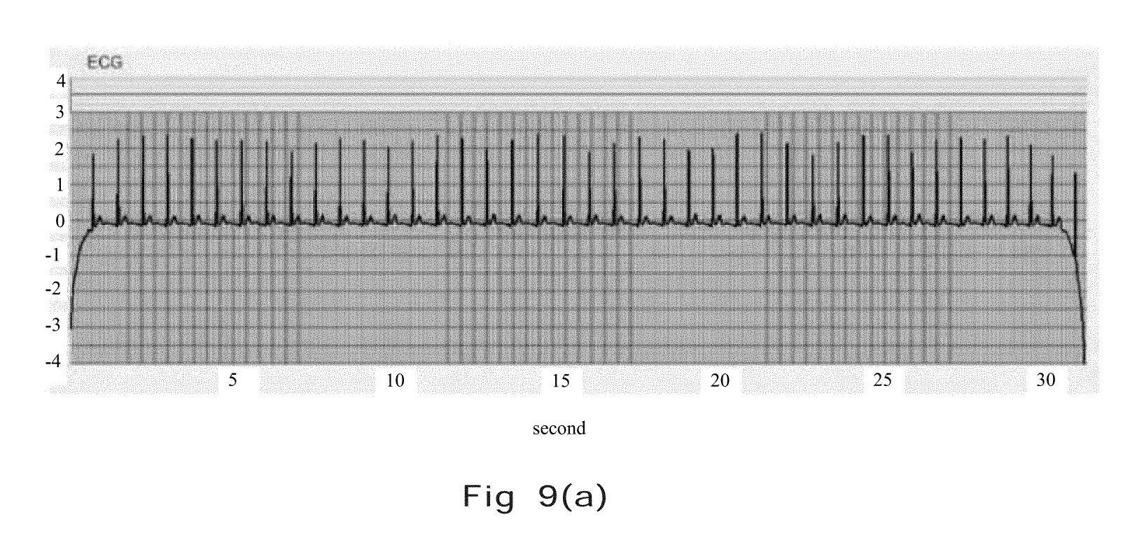

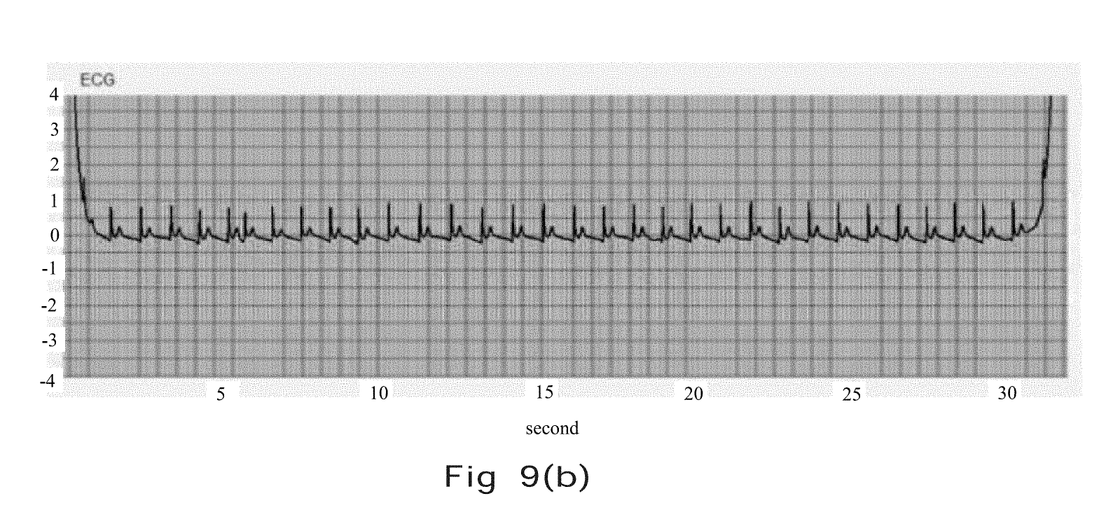

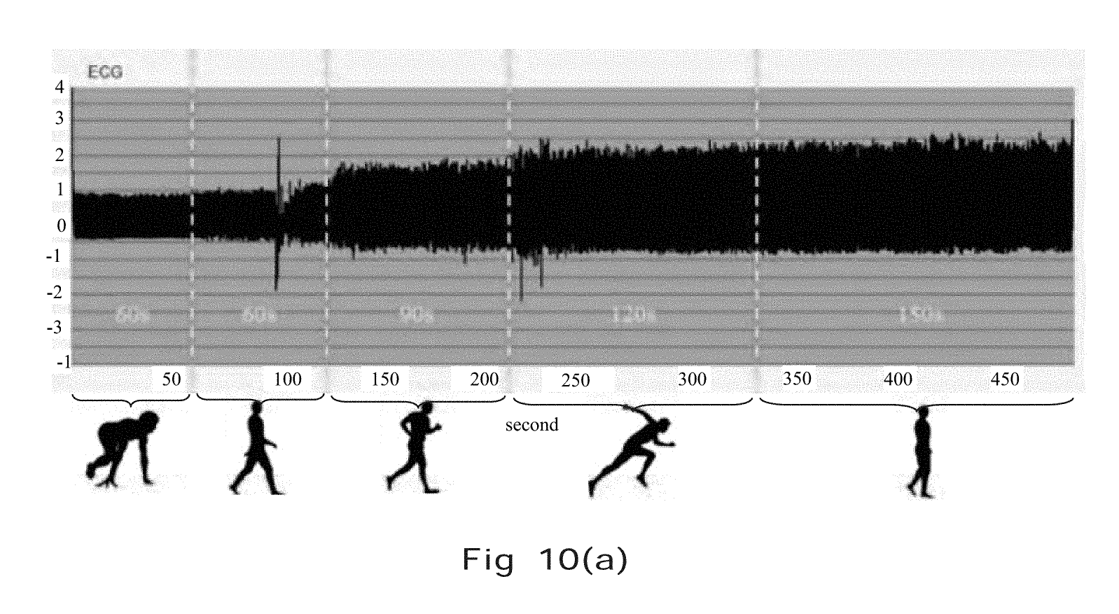

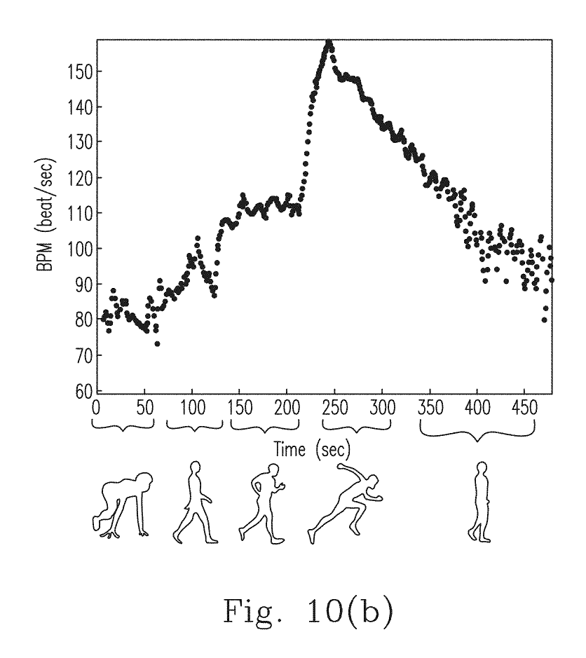

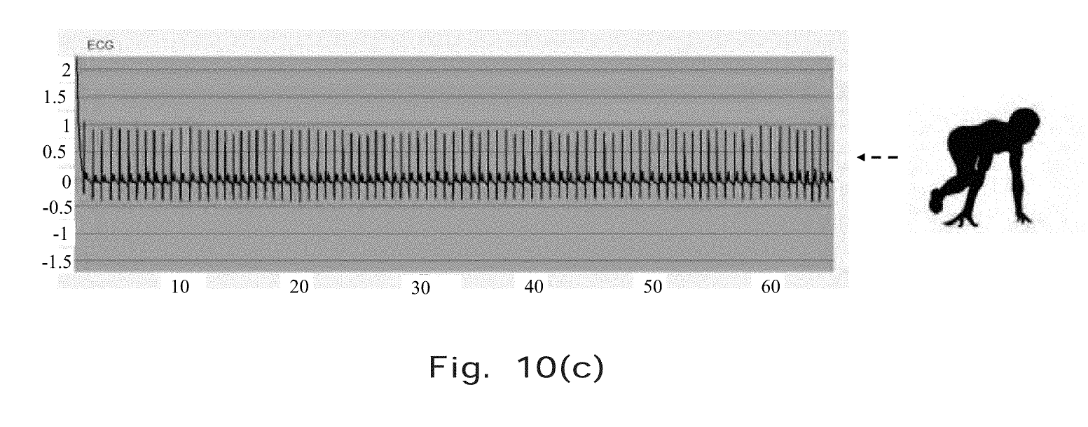

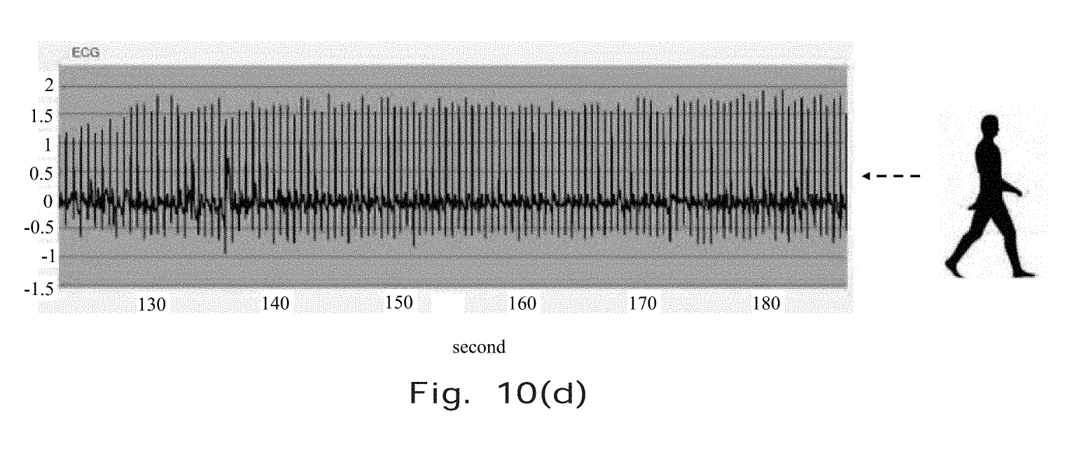

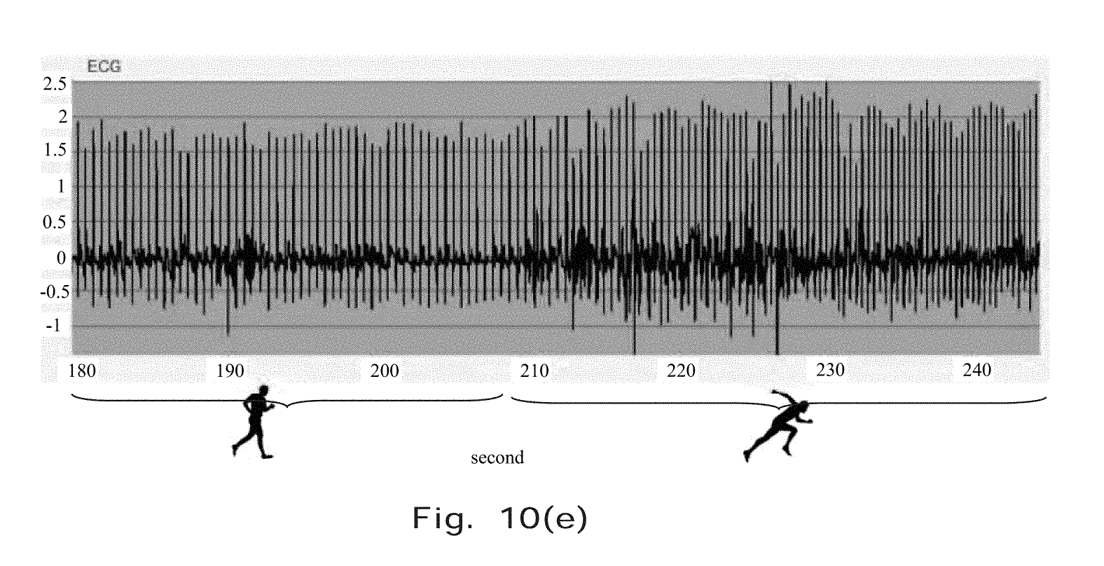

After washing fifty times, the electrode 5020 can maintain its sensed-intact signal of the ECG as shown in FIG. 9(a) and FIG. 9(b). Please refer to FIGS. 9(a) and 9(b). FIG. 9(a) shows an ECG signal of the carbon material 201 in a static condition; and FIG. 9(b) shows an ECG signal of the carbon-graphene material in the static condition. FIGS. 9(a) and (b) show that both ECG signals of the carbon material 201 and the carbon-graphene material in the static condition are stable after washing fifty times. The static condition means that a human's body does not move or do an activity. Furthermore, ECG signals measured in a dynamic condition are also steady after washing fifty times as shown in FIG. 10(a) to FIG. 10(e).

Please refer to FIG. 10(a), which shows an entire ECG of the electrode 5020 of the carbon-graphene material according to the preferred embodiment of the present disclosure. In FIG. 10(a), the horizontal axis represents the time in seconds, and the vertical axis represents the amplitude of the sensed voltage in micro volts. This preferred embodiment is illustrated by a 23 year-old man, who has a height and a weight being 165 cm and 55 kg respectively. From 0 to 60 seconds, the man is still in the static condition, but is walking, jogging and running during 61 to 120 seconds, 121 to 210 seconds, and 211 to 330 seconds respectively in the dynamic condition. From 331 to 480 seconds, the man rests. The resistance of the electrode 5020 of the carbon-graphene material is about 200±20 Ω/sq after washing fifty times.

Please refer to FIG. 10(b), which shows heart rates in the unit of beat/second according to the preferred embodiment of the present disclosure. It shows statuses of different heart rates while the man is still, walking, jogging and running.

Please refer to FIG. 10(c), FIG. 10(d) and FIG. 10(e). FIG. 10(c) shows an ECG signal when the man is still during 0 to 60 seconds. FIG. 10(d) shows an ECG signal while the man is walking during 120 to 180 seconds. FIG. 10(e) shows an ECG signal while the man is jogging and then running during 180 to 250 seconds.

### Embodiments

1. A sensing material for sensing a physiological parameter includes a carbon black material, a graphene material, and a glue material, wherein the carbon black material, the graphene material and the glue material are mixed together based on a specific weight ratio.

2. The material in Embodiment 1, wherein the sensing material further includes a nano carbon tube material. The carbon black material includes at least one of product types EC600JD and EC300J. The graphene material includes at least one selected from the group consisting of product types PML, PMF, PHF, TPGnP001, TPGnP002 and TPGnP003. The glue material includes at least one selected from the group consisting of polyurethane (PU), water polyurethane (WPU), thermoplastic PU, Silicone, Epoxy, rubber and plastisol.

3. The material of any one of Embodiments 1-2, wherein the specific weight ratio between the carbon black material and graphene material is 0˜2:2˜0, and the carbon black material and the graphene material have a combination weight percentage concentration wt % 5˜11 wt % in the glue material.

4. A method for producing a sensing material unit for sensing a physiological parameter, the method including steps of: providing a wearable object; providing a carbon black material, a graphene material and a glue material with a specific weight ratio thereamong; mixing the carbon black material, the graphene material and the glue material to form a mixed colloid; coating the mixed colloid onto the wearable object, drying the mixed colloid, and repeating the coating and drying steps until appropriate coating layers are formed so that the mixed colloid and the wearable object form a to-be-manufactured treated wearable object; and baking the to-be-manufactured treated wearable object to form the sensing material unit being in a nano scale and having an electrical parameter.

5. The method in one of Embodiment 4, wherein the process of forming the mixed colloid includes the following sub-steps: adding a water to mix with the glue material uniformly, wherein the water has an equal volume as that of the glue material; and stirring the water and the glue material under a room temperature for a specific period of time to form the mixed colloid by using a compound mulling machine, wherein the mixed colloid has a stickability dependent on the volume of the water.

6. The method of any one of Embodiments 4-5, wherein the mixed colloid is coated onto the wearable object by using a scraping knife, and the wearable object is a flexible fiber fabric.

7. The method of any one of Embodiments 4-6, wherein the to-be-manufactured treated wearable object has a surface blown by a heater.

8. The method of any one of Embodiments 4-7, further including steps of: placing the to-be-manufactured treated wearable object into a drying oven under a specific temperature and for a specific period of time; repeatedly washing the sensing material unit; and measuring the electrical parameter of the sensing material unit, wherein the electrical parameter includes at least a resistance value.

9. A unit for sensing a physiological parameter from an organism includes a nano carbon layer and a protective layer. The nano carbon layer transmits a physiological signal in response to the physiological parameter. The protective layer protects the nano carbon layer.

10. The unit in Embodiment 9, wherein the nano carbon layer includes: a nano carbon-mixture material; a sensing portion; and a transmission portion coupled to the sensing portion.

11. The unit of any one of Embodiments 9-10, wherein the transmission portion transmits the physiological signal; the sensing portion includes an electrode; and the transmission portion includes at least one of a wire and a wireless transceiver including an RFID transceiver.

12. The unit of any one of Embodiments 9-11, wherein the protective layer has at least one characteristic of water-isolation and electricity-isolation to prevent the nano carbon layer from being destroyed, and includes a first protective layer and a second protective layer. One of the first protective layer and the second protective layer has an opening structure.

13. The unit of any one of Embodiments 9-12, wherein the sensing portion senses the physiological parameter through the opening structure.

14. The unit of any one of Embodiments 9-13, wherein the physiological parameter includes at least one selected from the group consisting of a heart rate, a heart rhythm, a breath rate, a blood pressure, a pulse, an electrocardiogram (ECG) parameter, a pace count, a strength of activity and a calorie consumption.

15. The unit of any one of Embodiments 9-14, wherein the nano carbon layer includes a nano carbon-mixture material. The nano carbon-mixture material includes a nano carbon material and a glue material, and the nano carbon material includes at least one selected from the group consisting of a carbon black material, a graphene material and a nano carbon tube material. The carbon black material, the graphene material and the glue material ratio are mixed together with a specific weight thereamong to form the nano carbon-mixture material.

16. The unit of any one of Embodiments 9-15, wherein the carbon black material includes at least one of product types EC600JD and EC300J. The graphene material includes at least one selected from the group consisting of product types PML, PMF, PHF, TPGnP001, TPGnp002 and TPGnP003. The glue material includes at least one selected from the group consisting of polyurethane (PU), water polyurethane (WPU), thermoplastic PU, Silicone, Epoxy, rubber and plastisol.

17. The unit of any one of Embodiments 9-16, wherein the nano carbon-mixture material has an electrical conductivity, a flexibility and a uniformity. The specific weight ratio of the nano carbon material in the nano carbon-mixture material determines the electrical conductivity of the nano carbon-mixture material. The specific weight ratio of the glue material in the nano carbon-mixture material determines the flexibility of the nano carbon-mixture material. The specific weight ratio of the carbon black material in the nano carbon-mixture material determines the uniformity of the nano carbon material.

18. The unit of any one of Embodiments 9-17, further including at least one of a fabric and a wearable unit, wherein: the fabric includes a smart fabric including at least one of a smart sleeping accommodation and a smart umbrella; and the wearable unit includes at least one selected form the group consisting of a smart wristband, a smart buckle, a smart watch, a smart cloth, a smart earphone, a smart glass, a smart diaper, a smart banding, a smart heart rate meter and a smart leather-made product.

19. The unit of any one of Embodiments 9-18, wherein the unit is washed by water to achieve a clean state. The nano carbon layer is a sensor unit. The nano carbon-mixture material has a resistance value less than 100 ohm under the clean state.

20. The unit of any one of Embodiments 9-19, further including a clothes body and a processing unit, wherein: the nano carbon layer is a sensor unit; the cloth body encloses the protective layer; and the processing unit is disposed on the cloth body, and receives the physiological signal from the sensor unit.

While the invention has been described in terms of what is presently considered to be the most practical and preferred embodiments, it is to be understood that the invention need not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A sensing material for sensing a physiological parameter, comprising:
a carbon black material;
a graphene material; and
a glue material, wherein the carbon black material, the graphene material and the glue material are mixed together based on a specific weight ratio.

2. The sensing material as claimed in claim 1, wherein:
the sensing material further comprises a nano carbon tube material;
the carbon black material includes at least one of product types EC600JD and EC300J;
the graphene material includes at least one selected from the group consisting of product types PML, PMF, PHF, TPGnP001, TPGnP002 and TPGnP003; and
the glue material includes at least one selected from the group consisting of polyurethane (PU), water polyurethane (WPU), thermoplastic PU, Silicone, Epoxy, rubber and plastisol.

3. The sensing material as claimed in claim 1, wherein the specific weight ratio between the carbon black material and graphene material is 0˜2:2˜0, and the carbon black material and the graphene material have a combination weight percentage concentration wt % 5-11 wt % in the glue material.

4. A method for producing a sensing material unit for sensing a physiological parameter, the method comprising steps of:
providing a wearable object;
providing a carbon black material, a graphene material and a glue material with a specific weight ratio thereamong;
mixing the carbon black material, the graphene material and the glue material to form a mixed colloid;
coating the mixed colloid onto the wearable object, drying the mixed colloid, and repeating the coating and drying steps until appropriate coating layers are formed so that the mixed colloid and the wearable object form a to-be-manufactured treated wearable object; and
baking the to-be-manufactured treated wearable object to form the sensing material unit being in a nano scale and having an electrical parameter.

5. The method as claimed in claim 4, wherein:
the process of forming the mixed colloid includes the following sub-steps:
adding a water to mix with the glue material uniformly, wherein the water has an equal volume as that of the glue material; and
stirring the water and the glue material under a room temperature for a specific period of time to form the mixed colloid by using a compound mulling machine, wherein the mixed colloid has a stickability dependent on the volume of the water.

6. The method as claimed in claim 4, wherein the mixed colloid is coated onto the wearable object by using a scraping knife, and the wearable object is a flexible fiber fabric.

7. The method as claimed in claim 4, wherein the to-be-manufactured treated wearable object has a surface blown by a heater.

8. The method as claimed in claim 4, further comprising steps of:
placing the to-be-worked manufactured wearable object into a drying oven under a specific temperature and for a specific period of time;
repeatedly washing the sensing material unit; and
measuring the electrical parameter of the sensing material unit, wherein the electrical parameter includes at least a resistance value.

9. A unit for sensing a physiological parameter from an organism, comprising:
a nano carbon layer transmitting a physiological signal in response to the physiological parameter; and
a protective layer protecting the nano carbon layer.

10. The unit as claimed in claim 9, wherein the nano carbon layer includes:
a nano carbon-mixture material;
a sensing portion; and
a transmission portion coupled to the sensing portion.

11. The unit as claimed in claim 10, wherein:
the transmission portion transmits the physiological signal;
the sensing portion includes an electrode; and
the transmission portion includes at least one of a wire and a wireless transceiver including an RFID transceiver.

12. The unit as claimed in claim 9, wherein:
the protective layer has at least one characteristic of water-isolation and electricity-isolation to prevent the nano carbon layer from being destroyed, and includes a first protective layer and a second protective layer; and
one of the first protective layer and the second protective layer has an opening structure.

13. The unit as claimed in claim 12, wherein the sensing portion senses the physiological parameter through the opening structure.

14. The unit as claimed in claim 9, wherein the physiological parameter includes at least one selected from the group consisting of a heart rate, a heart rhythm, a breath rate, a blood pressure, a pulse, an electrocardiogram (ECG) parameter, a pace count, a strength of activity and a calorie consumption.

15. The unit as claimed in claim 9, wherein:
the nano carbon layer includes a nano carbon-mixture material;
the nano carbon-mixture material includes a nano carbon material and a glue material, and the nano carbon material includes at least one selected from the group consisting of a carbon black material, a graphene material and a nano carbon tube material;
the carbon black material, the graphene material and the glue material ratio are mixed together with a specific weight thereamong to form the nano carbon-mixture material.

16. The unit as claimed in claim 15, wherein:
the carbon black material includes at least one of product types EC600JD and EC300J;
the graphene material includes at least one selected from the group consisting of product types PML, PMF, PHF, TPGnP001, TPGnp002 and TPGnP003; and
the glue material includes at least one selected from the group consisting of polyurethane (PU), water polyurethane (WPU), thermoplastic PU, Silicone, Epoxy, rubber and plastisol.

17. The unit as claimed in claim 15, wherein:
the nano carbon-mixture material has an electrical conductivity, a flexibility and a uniformity;
the specific weight ratio of the nano carbon material in the nano carbon-mixture material determines the electrical conductivity of the nano carbon-mixture material;
the specific weight ratio of the glue material in the nano carbon-mixture material determines the flexibility of the nano carbon-mixture material; and
the specific weight ratio of the carbon black material in the nano carbon-mixture material determines the uniformity of the nano carbon material.

18. The unit as claimed in claim 9, further comprising at least one of a fabric and a wearable unit, wherein:
the fabric includes a smart fabric including at least one of a smart sleeping accommodation and a smart umbrella; and
the wearable unit includes at least one selected form the group consisting of a smart wristband, a smart buckle, a smart watch, a smart cloth, a smart earphone, a smart glass, a smart diaper, a smart banding, a smart heart rate meter and a smart leather-made product.

19. The unit as claimed in claim 9, wherein:
the unit is washed by water to achieve a clean state;
the nano carbon layer is a sensor unit; and
the nano carbon-mixture material has a resistance value less than 100 ohm under the clean state.

20. The unit as claimed in claim 9, further comprising a cloth body and a processing unit, wherein:
the nano carbon layer is a sensor unit;
the cloth body encloses the protective layer; and
the processing unit is disposed on the cloth body, and receives the physiological signal from the sensor unit.
